(19) United States
(12) Patent Application Publication
(10) Pub. No.: US 2025/0382244 A1
(43) Pub. Date: Dec. 18, 2025

(54) ELECTRICALLY HEATED FURNACES UTILIZING CONDUCTIVE REFRACTORY MATERIALS

(71) Applicant: SABIC Global Technologies B.V., Bergen op Zoom (NL)

(72) Inventors: Michael Edward Huckman, Bengaluru (IN); Joseph William Schroer, Bengaluru (IN); Robert Broekhuis, Bengaluru (IN)

(21) Appl. No.: 18/867,560

(22) PCT Filed: May 25, 2023
(86) PCT No.: PCT/EP2023/064098

§ 371 Date: Nov. 20, 2024

(30) Foreign Application Priority Data
May 27, 2022 (EP) 22175712.3

(51) Int. Cl. C07C 4/04; B01J 19/00; B01J 19/24; C01B 3/34; F27D 11/02; H05B 3/14; H05B 3/62

(52) U.S. Cl. CPC C07C 4/04; B01J 19/0013; B01J 19/2415; C01B 3/342; F27D 11/02; H05B 3/141; H05B 3/62; B01J 2219/00135; C01B 2203/0205; C01B 2203/085; C01B 2203/1241; H05B 2214/03

(57) ABSTRACT

Systems and methods are described for electrically heated chemical processes utilizing conductive refractory materials. A heating apparatus may include a conductive refractory material without separate heating elements; and a furnace for heating hydrocarbons. The furnace includes one or more process tubes that are configured to receive a process vapor or fluid such that the process vapor or fluid does not contact the conductive refractory material. The conductive refractory material may be at least partially disposed within the furnace and configured to receive electrical power from a power source and to generate heat such that the conductive refractory material directly radiates heat within the furnace. A method of operating a chemical process may include providing such a furnace; and applying electricity directly to the conductive refractory material such that the conductive refractory material increases in temperature and provides heat to a chemical process.

### CROSS-REFERENCE TO RELATED APPLICATIONS

This application claims the benefit of European Patent Application No. EP22175712, filed 27 May 2022, which is incorporated by reference herein in its entirety.

### FIELD OF DISCLOSURE

The present disclosure relates to systems and methods for generating thermal energy and, more particularly, to heating devices for electrically heated processes.

### BACKGROUND

High temperature furnaces are useful for various applications, including, but not limited to chemical processes. In particular, radiative high temperature furnaces have been contemplated for steam cracking, steam methane reforming (SMR), reforming for ammonia, dehydrogenation, tar cracking, or similar applications. Such processes and furnaces for heating such processes traditionally rely on use of combustion. To meet new sustainability and carbon dioxide emission reduction requirements such fired heaters may need to be replaced.

Recent developments have included heaters and furnaces powered by electricity, preferably renewable electricity. Non-combustion heaters are needed to provide heat to such processes. Exemplary constructions of heaters for such applications have included a conductive heating element, often a metal wire or metal ribbon as in FIGS. 1A and 1B hung on a non-conductive refractory material, such as a non-conductive refractory brick. In these systems, voltage is applied across the conductive element and the resulting current causes the element to heat up. This is called impedance or ohmic heating. As the elements heat up, they heat the refractory. As the system reaches operating temperature the heater radiates heat from the elements and from the refractory brick out into the furnace box where heat is transferred to furnace tubes. This allows for substitution of combustion with electrically heated systems.

FIGS. 1A and 1B schematically illustrate a conventional radiative heating element 100 that may be used for heating applications, such as heating hydrocarbons. Metal, ceramic, or other types of material heating elements (e.g., wires or ribbons 102) may be attached to non-conductive refractory materials 104, i.e., the heating elements are not integral with the non-conductive refractory materials 104. When the wires or ribbons 102 are heated, the thermal energy generated by the heating elements 102 heats the non-conductive refractory material 104, and heat radiates from the heating elements and the non-conductive refractory material 104 toward the interior of a furnace enclosure.

In the above described systems, however, the allowable operating temperature of the heating elements is limited and lifetime decreases as temperature increases. The operating maximum temperatures may vary, but in general, for metal elements the operating maximum temperatures should not exceed approximately 1300° C. In many processes, however, higher temperatures are required.

For electrically powered processes, the inventors have identified a need for systems and methods for heating using heaters that are able to operate at high temperatures.

### SUMMARY

In an embodiment of the present disclosure, a heating apparatus may include a conductive refractory material without separate heating elements (and non-conductive refractory material); and a furnace for heating hydrocarbons. The furnace may comprise one or more process tubes extending through an interior of the furnace and configured to receive a process vapor or fluid such that the process vapor or fluid does not contact the conductive refractory material. The conductive refractory material may be at least partially exposed to an interior of (e.g., at least partially disposed within) the furnace, and may be configured to receive electrical power from a power source to generate heat such that the conductive refractory material radiates the heat (e.g., directly) to the interior of the furnace.

In an embodiment of the present disclosure, a method of operating a chemical process may include: providing a furnace with a conductive refractory material that is at least partially exposed to (e.g., disposed within) an interior of the furnace. The furnace may comprise one or more process tubes extending through the interior of the furnace and configured to receive a process vapor or fluid such that the process vapor or fluid does not contact the conductive refractory material. The method may further include applying electricity directly to the conductive refractory material such that electrical current flowing through the conductive refractory material generates thermal energy to increase the temperature of the conductive refractive material and heat a process vapor or fluid in the process tubes.

As used in this disclosure, “conductive refractory material” is a material that itself conducts electricity such that electrical current flowing through the material itself generates thermal energy (e.g., converts electrical energy to thermal energy)—i.e., the conductive refractory material does not include separate heating elements. Various examples of conductive refractory materials are known and commercially available, such as, for example, the ETS Joule Hive Technology available from Electrified Thermal Solutions, Inc. (Medford, Massachusetts, USA).

In certain embodiments, the conductive refractory material may be a ceramic material (e.g., an electrically heated ceramic material). The one or more process tubes of the furnace may be configured to receive the process vapor or fluid associated with a process selected from the group consisting of steam cracking, steam methane reforming, reforming for ammonia, dehydrogenation, and tar cracking. The one or more process tubes may comprise a plurality of process tubes. In some aspects, the plurality of process tubes may be arranged in rows of two or more process tubes and said rows may alternate with the conductive refractory material. Alternatively, in some aspects, the plurality of process tubes may be arranged in a single row of multiple process tubes. In such cases, each individual process tube of the plurality of process tubes may be separated from the conductive refractory material by at least a process tube wall thickness corresponding to a difference between a process tube outer diameter and a process tube inner diameter. The conductive refractory material may be shaped as cylinders, plates, or rods. The conductive refractory material may form channels. According to certain embodiments, the one or more process tubes include a plurality of process tubes, and each individual process tube is disposed within a particular channel. To illustrate, in some cases, the conductive refractory material includes a plurality of channels extending through the conductive refractory material, and each individual process tube may be inserted through a corresponding one of the plurality of channels. The electrical power from the power source may correspond to a voltage greater than 500 volts, preferably greater than 1000 volts, and more preferably greater than 2000 volts. In an embodiment of the present disclosure, a method of operating a chemical process may include providing a furnace with a conductive refractory material that is at least partially disposed within the furnace, the conductive refractory material without separate heating elements, wherein the furnace comprises one or more process tubes configured to receive a process vapor or fluid, the one or more process tubes separated from the conductive refractory material to prevent the process vapor or fluid from directly contacting the conductive refractory material; and applying electricity directly to the conductive refractory material such that the conductive refractory material increases in temperature and provides heat to a chemical process.

In certain embodiments, the chemical process may correspond to one of: a steam cracking process (e.g., an ethane (C₂H₆) cracking process or a steam cracking for olefins process, in some examples); a steam methane reforming (SMR) process (e.g., for syngas production); a reforming for ammonia, hydrogen, or methanol process; a dehydrogenation process (e.g., of propane to propylene); and a tar cracking process. According to certain embodiments, the chemical process may be a steam methane reforming process, and the one or more process tubes may comprise a plurality of process tubes. According to certain embodiments, the chemical process may be an ethane cracking process or a steam cracking for olefins process, and the one or more process tubes may comprise a plurality of process tubes. In such cases, each individual process tube of the plurality of process tubes may be separated from the conductive refractory material by at least a process tube wall thickness corresponding to a difference between a process tube outer diameter and a process tube inner diameter.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Some details associated with the aspects of the present disclosure are described above, and others are described below. Other implementations, advantages, and features of the present disclosure will become apparent after review of the entire application, including the Brief Description of the Drawings, Detailed Description, and the Claims.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a schematic front view of a conventional radiative heating element that may be used for heating applications.

FIG. 1B is a schematic side view of a conventional radiative heating element that may be used for heating applications.

FIG. 2 is a schematic view of an exemplary conductive refractory system.

FIG. 3A is a flow diagram of conductive refractory systems used as a heat storage device.

FIG. 3B is a flow diagram of conductive refractory systems used as a furnace.

FIGS. 4A and 4B, respectively, are side and top view schematic diagrams showing use of a stack of conductive refractory materials to heat process tubes.

FIGS. 5A and 5B, respectively, are side and end view schematic diagrams showing use of walls of conductive refractory materials alternating with rows of process tubes.

FIG. 6 is a schematic diagram showing a cutaway side view of an example of a furnace design that includes multiple process tubes disposed between two walls of conductive refractory material.

FIG. 7 is a graph depicting modeling results corresponding to tube temperature profiles associated with an example SMR process performed utilizing a furnace design similar to the furnace design depicted in FIG. 6.

FIG. 8 is a schematic diagram showing a cutaway side view of another example of a furnace design that includes multiple process tubes disposed between two walls of conductive refractory material.

FIG. 9 is a graph depicting modeling results corresponding to tube temperature profiles associated with an example ethane cracking process performed utilizing a furnace design similar to the furnace design depicted in FIG. 8.

### DETAILED DESCRIPTION

The drawings include like numerals to indicate like parts throughout the several views, the following description is provided as an enabling teaching of exemplary embodiments, and those skilled in the relevant art will recognize that many changes may be made to the embodiments described. It also will be apparent that some of the desired benefits of the embodiments described may be obtained by selecting some of the features of the embodiments without utilizing other features. Accordingly, those skilled in the art will recognize that many modifications and adaptations to the embodiments described are possible and may even be desirable in certain circumstances. Thus, the following description is provided as illustrative of the principles of the embodiments and not in limitation thereof.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to,” unless otherwise stated. Thus, the use of such terms is meant to encompass the items listed thereafter, and equivalents thereof, as well as additional items. The transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

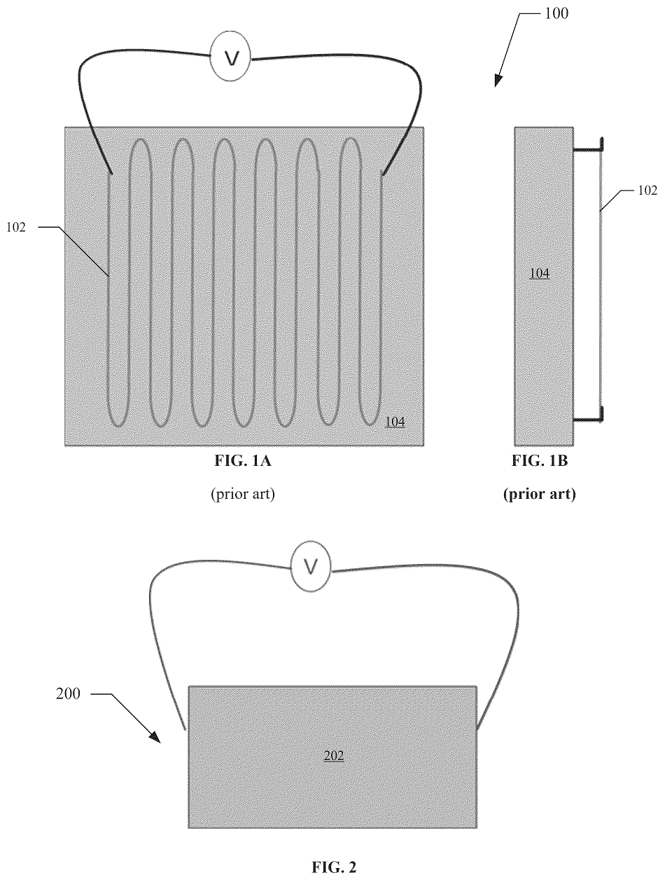

In contrast to the prior art configurations of FIGS. 1A and 1B, FIG. 2 shows an example of an exemplary conductive refractory system 200 comprising a conductive refractory material (e.g., in the form of bricks) that is configured to pass electric current for resistive heating. In some configurations, the conductive refractory bricks include a conductive material or may be doped with a conductive material (e.g., metal oxide) such that electrical current will flow through the brick, but with sufficient resistance to convert electrical energy to thermal energy and thereby heat the material. Such conductive refractory materials may have both a relatively high emissivity (e.g., 0.5 or greater, 0.7 or greater, or 0.8 or greater) for efficient emission of thermal radiation and relatively high specific heat (e.g., 400 J/kg-K or greater, 450 J/kg-K or greater, or 500 J/kg-K or greater) to increase the thermal energy to temporarily “store” as thermal energy is dissipated (e.g., in the event that electric current is no longer provided).

The use of conductive refractory material allows the stack to omit conventional heating metal elements, such as wires or ribbons, which are typically more susceptible to oxidation and other degradation at elevated temperatures (and are therefore not suitable for use at temperatures comparable to those produced by conventional burners. In use, the conductive refractory material of conductive refractory system is heated by applying electricity directly to the conductive refractory material 202.

The conductive refractory materials of the present disclosure may be configured to pass electric current for resistive heating. The use of conductive refractory materials, such as refractory bricks, may provide an alternative to the metal wire or metal ribbon heating elements, or separate ceramic or other types of heating elements that are not integrated components of a conductive refractory system, such as silicon carbide (SiC) or molybdenum carbide (MoC) heating elements. For purposes of this application, reference may be made to “bricks”, but the conductive refractory material of the system can be provided in various formats, sizes, shapes, and other configurations depending on the needs of particular embodiments.

The use of conductive refractory materials may further eliminate or reduce certain design constraints of those existing systems. In general, the conductive refractory materials may be bricks with no metal conducts but instead current flows through the conductive refractory materials. In certain embodiments, the conductive refractory material may be ceramic. In still other embodiments, the conductive refractory material may be a standalone device that does not include separate heating elements. In certain embodiments, the conductive refractory materials may also be referred to as electrically heated ceramics (EHCs).

In some configurations, conductive refractory materials of the present disclosure may be configured to operate at temperatures up to 2000° C. or greater and much higher than the operating temperatures of conventional electrical heating elements (e.g., Kanthal-FeCrAl alloys or Fe-like, Cr-like, Al-like metallic elements or alloys thereof).

Advantages to the present conductive refractory systems can include, but are not limited to, one or more (e.g., a combination of any two or more) of the following:
Improved simplicity and cost. Conductive refractory materials need not utilize separate conductive wires or other heating elements of various materials (metal, ceramic, etc.), so the use of conductive refractory materials can for the present conductive refractory systems eliminate such separate conductive wires or other heating elements, which are one of the most expensive components of the heating technology. Put more concisely, the elimination of separate conductive wires or other heating elements allows for simpler conductive refractory systems.
Higher heat flux and higher temperatures. Conductive refractory materials may operate at temperatures as high as approximately 2000 C, whereas metal heating elements are limited to approximately 1300 C. Conductive refractory materials may have improved maintenance and reliability compared to metal wires and metal ribbons, both because they need not be operating near temperature limits of the materials and because they may be simpler in structure (reducing the potential points of failure within a system).
The conductive refractory systems may be used to heat to higher temperatures.
The conductive refractory materials may attain higher (total wall) fluxes. To illustrate, there may be two dimensions to increasing flux: (1) at higher temperatures, the flux may be higher; and (2) utilizing a greater fraction of the total wall area. As a heated refractory system can utilize a greater portion of the wall area of the conductive refractory material, the required total surface area and furnace size may be reduced (relative to traditional electrically heated refractories) for a given duty.
Higher Voltage. Conductive refractory systems may operate at voltages as high as 13 kV. In certain embodiments, the electricity may be provided at a voltage greater than 500 volts, preferable greater than 1000 volts, and more preferably greater than 2000 volts. Therefore, heating may be achieved at much lower amperage, and thus the required electrical equipment may be significantly less expensive. Electrical equipment may include, but is not limited to, step down transformers, control elements, switch gear, conductors, connections, and other devices.

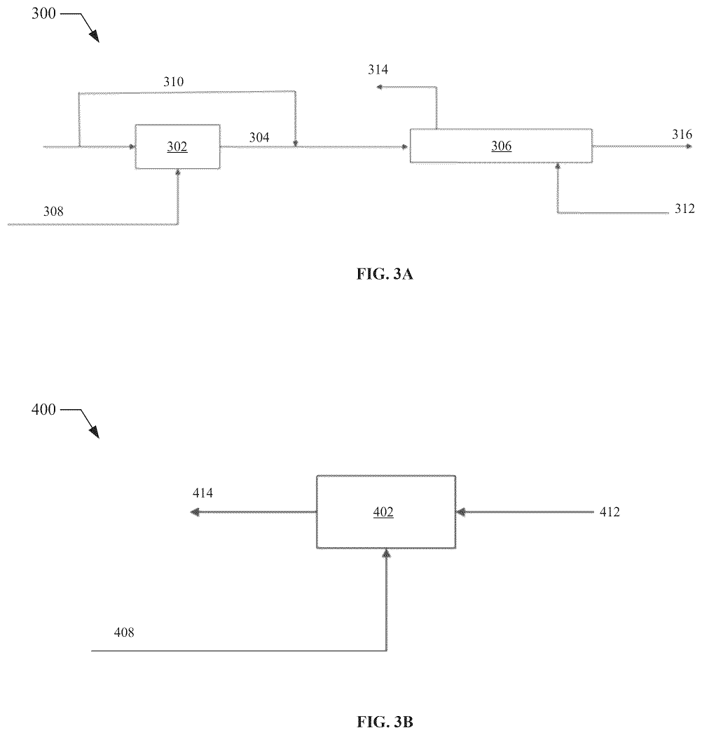

Energy storage technologies may provide heat or thermal energy to process streams via conductive refractory systems. The heat may be used for heating itself or to drive chemistry in various chemical processes. FIG. 3A is a flow diagram 300 of conductive refractory systems used as a heat storage device. In certain embodiments, where the conductive refractory material serves as a heat storage device, heat would be drawn from a conductive refractory system 302, such as one comprising or structured of bricks of conductive refractory bricks (e.g., a heat storage brick stack, according to some embodiments) arranged within a furnace or other area to be heated. Heat from the conductive refractory system 302 may be utilized to create a hot working fluid 304 (usually a hot gas) that then delivers the thermal energy to a process, such as, via a heat exchanger 306. The flow diagram 300 of FIG. 3A further illustrates that the conductive refractory system 302 may include a power input to receive power 308 (e.g., from an alternating current (AC) power source, in some cases). The flow diagram 300 further illustrates that, according to some aspects of the present disclosure, a bypass 310 (e.g., a gas bypass, in some cases) may be configured to direct a portion of working fluid (e.g., a gas) to bypass conductive refractory system 302 and be re-directed to join the hot working fluid 304 downstream of a working fluid output of the conductive refractory system 302.

The flow diagram 300 of FIG. 3A further illustrates that heat exchanger 306 may further include at least one process input and at least one process output. The process input(s) may be configured to receive a process feed 312, and heat exchanger 306 may heat the process feed to form a hot process output 314 that is output via the process output(s). The flow diagram 300 of FIG. 3A further illustrates that heat exchange reactor 306 may further include at least one working fluid output configured to output a reduced temperature working fluid 316 after thermal energy is transferred to process feed 312 to produce the hot process output 314.

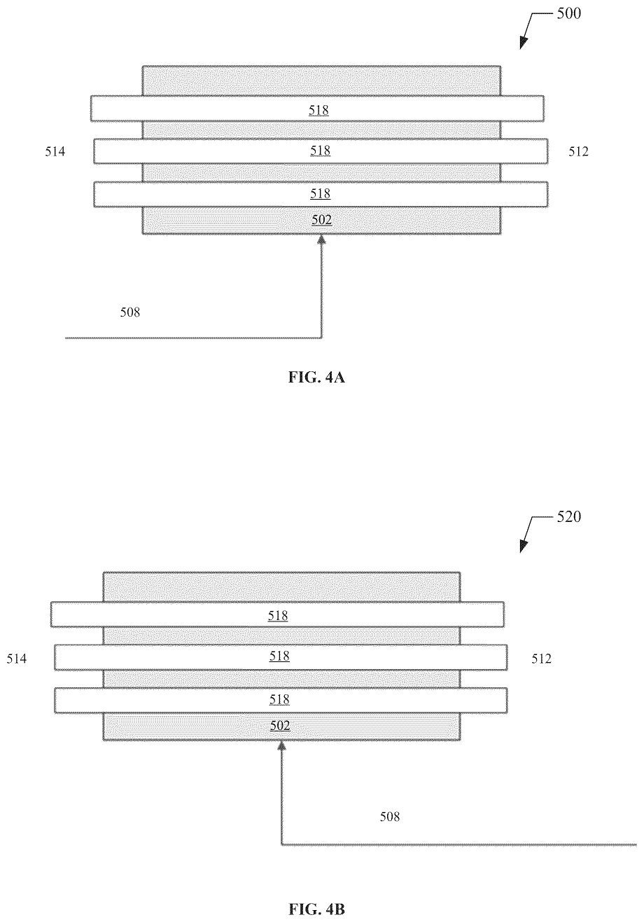

FIG. 3B is a flow diagram 400 of conductive refractory systems used as a furnace. In certain embodiments, when the conductive refractory material is used as a furnace (referred to herein as either a “conductive refractory system” or “conductive brick furnace” and identified by reference character 402 in FIG. 4B), heat may be delivered directly from hot bricks in the same way as with conventional technology, but without the limitations of the conductive metal wires or metal ribbons. Similar to the flow diagram 300 of FIG. 3A depicting the example conductive refractory system 302, the flow diagram 400 of FIG. 3B further illustrates that the conductive refractory system 402 may include a power input to receive power 408 (e.g., from an alternating current (AC) power source, in some cases).

FIG. 3B further illustrates that, in some cases, the conductive refractory system 402 may further include at least one process input and at least one process output. The process input(s) may be configured to receive a process feed 412, and the conductive refractory system 402 may directly apply heat (e.g., directly from the hot bricks, corresponding to a temperature of approximately 1000° C., according to one embodiment) to create a hot process output 414 that is output via the process output(s). As an illustrative, non-limiting example, an initial temperature of the process feed 412 (e.g., a hydrocarbon feed, in some cases) at the process input(s) of the conductive refractory system 402 may be approximately 650° C., and an output temperature of the hot process output 414 at the process output(s) of the conductive refractory system 402 may be approximately 850° C. after application of the heat.

In certain embodiments, the conductive refractory systems include process tubes passing or extending through a stack of conductive refractory material. For example, FIGS. 4A and 4B are diagrams 500 and 520 depicting side and top schematic views, respectively, showing use of a conductive refractory material 502 (or a stack of conductive refractory materials) to heat process tubes 518, showing process feed 512 (e.g., a hydrocarbon feed) at inputs to each of the individual tubes and hot process output 514 at outputs of each of the individual tubes.

In certain embodiments, the process tubes 518 may penetrate or otherwise pass through the conductive refractory material 502. The conductive refractory material 502 may have one or more passages (also referred to herein as “channels” or “tunnels”) through which one or more process tubes 518 may be inserted. In such instances, the one or more process tubes 518 may be partially or completely surrounded by the conductive refractory material 502. In certain embodiments, the conductive refractory material 502 may be a stack. Power 508 input may be ohmic heating as a voltage is applied across the stack. Heat may be radiatively transferred from hot bricks (may be as hot as approximately 2000 C) to the process tubes 518, such as hydrocarbon tubes, and then to the material within the process tubes 518, such as hydrocarbon gas (e.g., received via the process feed 512 at inputs to the individual tubes 518 and output via the hot process output 514 at outputs of the individual tubes 518).

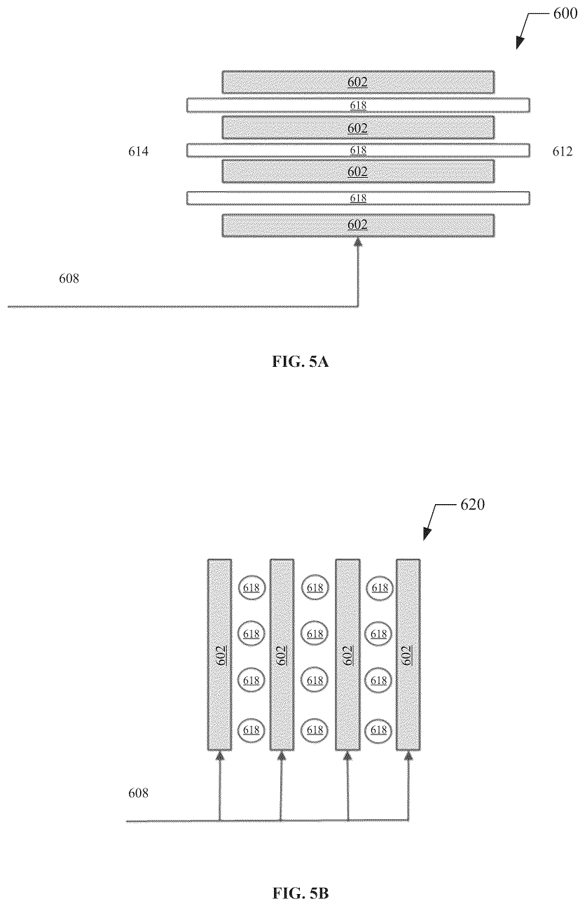

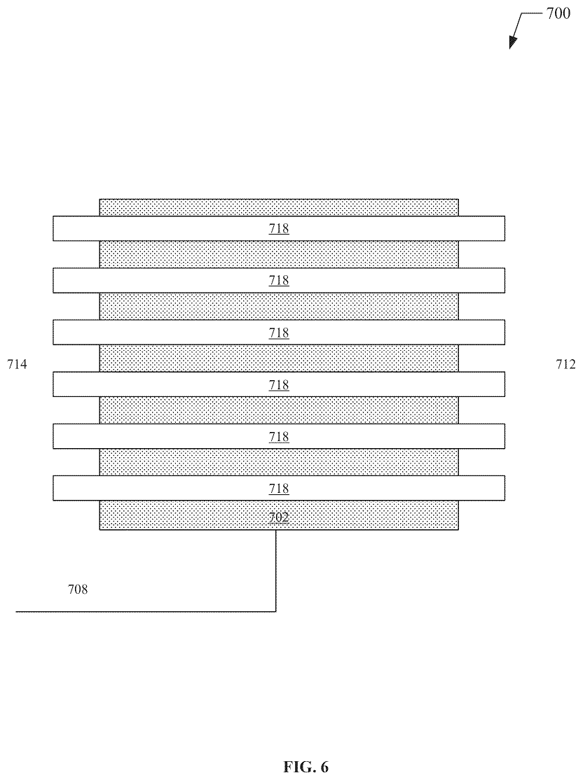

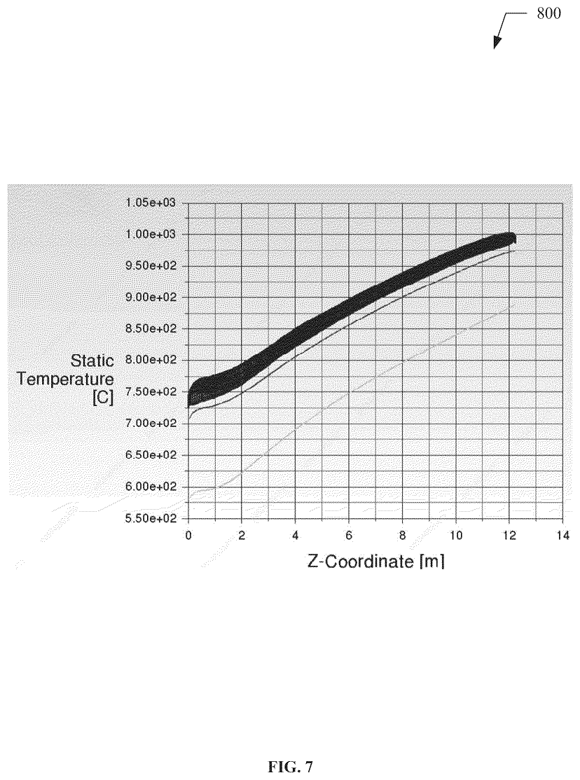

In certain embodiments, the conductive refractory systems may include alternating walls of conductive refractory material and rows of process tubes. FIGS. 5A and 5B are schematic diagrams 600 and 620 showing use of walls of conductive refractory materials alternating with rows of process tubes 618, showing process feed 612 (e.g., a hydrocarbon feed) at inputs to each of the individual tubes 618 and hot process output 614 at outputs of each of the individual tubes 618. The schematic diagrams 600 and 620 depicted in FIGS. 5A and 5B correspond to a top view and an end view, respectively, showing use of a stack of conductive refractory materials 602 to heat the process tubes 618.

In certain embodiments, conductive refractory systems may include alternating brick walls and rows of process tubes. In general, the size and configuration of the conductive refractory materials may be varied to provide the required heat for specific applications. In certain embodiments, walls may include one or more bricks of conductive refractory material and may be one or more bricks in width.

In certain embodiments, the wall may be a stack of conductive and non-conductive refractory bricks. Power input may be ohmic heating as a voltage is applied across the stack. Heat may be radiatively transferred from a hot brick wall (may be as hot as approximately 2000° C.) to the process tubes, such as hydrocarbon tubes, and then to the material within the tubes, such as hydrocarbon gas.

In addition, various other geometries are possible. The examples set forth in FIGS. 4A-4B and 5A-5B are illustrative geometries only and other configurations are possible to provide heat to various processes. Exemplary configurations may include, but are not limited to:
Process tubes may be within a box with the heat generating conductive refractory materials (powered by electricity) lined on the wall. Non-conductive refractory materials may also be used to line the outer wall to insulate it from a (e.g., metal) container wall and ambient conditions.
Multiple rows of tubes and conductive refractory material in alternating patterns.
Conductive refractory materials in various geometries including, but not limited to, cylinders, plates, etc. to construct high temperature resilient heating elements or structures channels or structures porous network frameworks.

The systems and methods described herein may be used to heat a process stream or to drive endothermic chemistry with or without a catalyst.

### Example 1

Referring to FIG. 6, a schematic diagram 700 shows a cutaway side view of an example of a furnace having a design similar in some respects to the one described herein with respect to FIGS. 5A and 5B. The example furnace design depicted in FIG. 6 includes multiple process tubes 718 disposed between two walls (only one of which is shown) of a conductive refractory material 702 to prevent process vapor or fluid from directly contacting the conductive refractory material 702.

In the example of FIG. 6, the multiple process tubes 718 correspond to six different process tubes arranged in a single row, where the single row is situated between the two walls of the conductive refractory material 702. Each of the individual process tubes 718 were modeled with the following properties: a tube inner diameter (ID) of 127 mm; a tube length of 12.22 m; and a tube wall thickness of 10 mm. The overall surface area of the outer surface of all of the process tubes 718 exposed to radiative heating between the two walls of the conductive refractory material 702 corresponded to 70 meters squared.

The example furnace design depicted in FIG. 6 was modeled for a steam methane reforming (SMR) process. Power 708 provided to the conductive refractory material 702 corresponded to an energy input of 0.4 MW for each of the individual process tubes 718. Tube inlet feed conditions are depicted below in Tables 1 and 2. Tube outlet conditions (corresponding to variables averaged for each of the individual process tubes 718) are depicted below in Table 3.

TABLE 1

| | |
|---|---|
| Temperature (° C.) | 565 |
| Flow rate (kg/s) per tube | 0.124089 |
| Pressure (Pascals) | 4175000 |

TABLE 2

| Species: | Mass fraction: |
|---|---|
| C2H6 | 0.01769 |
| H2 | 0.00137 |
| H2O | 0.76209 |
| CO2 | 0.00174 |
| CH4 | 0.1987 |
| N2 | 0.01077 |
| C3H8 | 0.00748 |

TABLE 3

| | |
|---|---|
| Temperature (K) | 1161.327 |
| Wall Temperature (K) | 1246.8271 |
| Nunnselt Number | 617.65716 |
| Wall Heat Flux (W/m2) | −63929.765 |
| Velocity (m/s) | 1.8351336 |
| Density (kg/m3) | 5.3439704 |
| Pressure (Pascals) | 4175000 |
| Mass fraction of C2H6 | 0 |
| Mass fraction of H2 | 0.0770734 |
| Mass fraction of H2O | 0.48162041 |
| Mass fraction of CO2 | 0.18272876 |
| Mass fraction of CH4 | 0.041943179 |
| Mass fraction of N2 | 0.010772998 |
| Mass fraction of C3H8 | 0 |
| Mass fraction of CO | 0.20586125 |

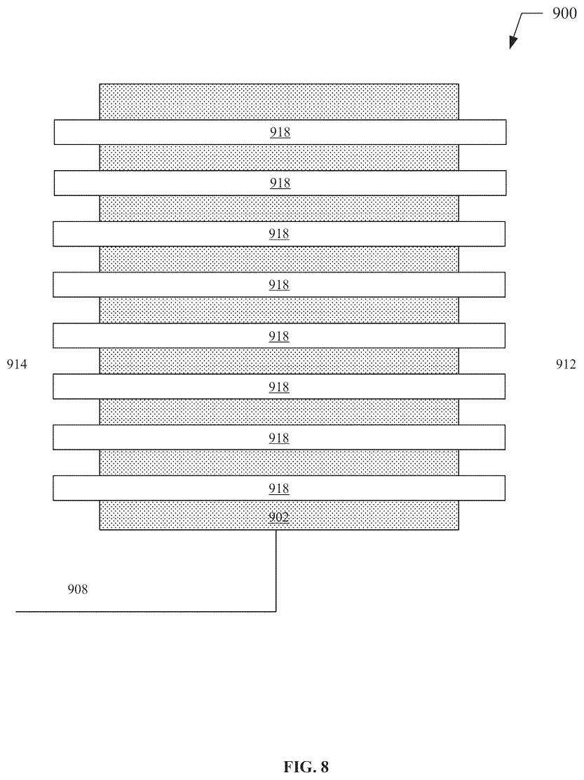

Based on a comparison of the initial mass fraction of methane (CH₄) as shown in Table 2 to the mass fraction of methane as shown in Table 3, the SMR process modeling with the furnace design depicted in FIG. 6 resulted in a methane conversion rate of about 79 percent. With respect to thermal data, flux at the heating wall corresponded to 33.55 kW/m² (averaged based on wall area); and flux at the tube wall corresponded to 80.7 kW/m² (averaged based on identifiers associated with each of the individual process tubes 718). With respect to wall temperature data, a maximum wall temperature modeled was about 1215° C. and an average wall temperature measurement was about 1151° C. FIG. 7 is a graph 800 depicting tube temperature profiles, where the top line (“thick”) corresponds to a temperature at the outer diameter (T_OD) of the process tubes 718, the middle line corresponds to a temperature at the inner diameter (T_ID) of the process tubes 718, and the bottom line corresponds to a temperature of the process gas (T_gas) within the process tubes 718. The “thickness” of the top line in the graph 800 depicted in FIG. 7 is because there are a range of temperatures around the circumference of each of the individual process tubes 718, with the hottest temperatures facing the heating walls and with the coolest temperatures facing adjacent tube(s).

### Example 2

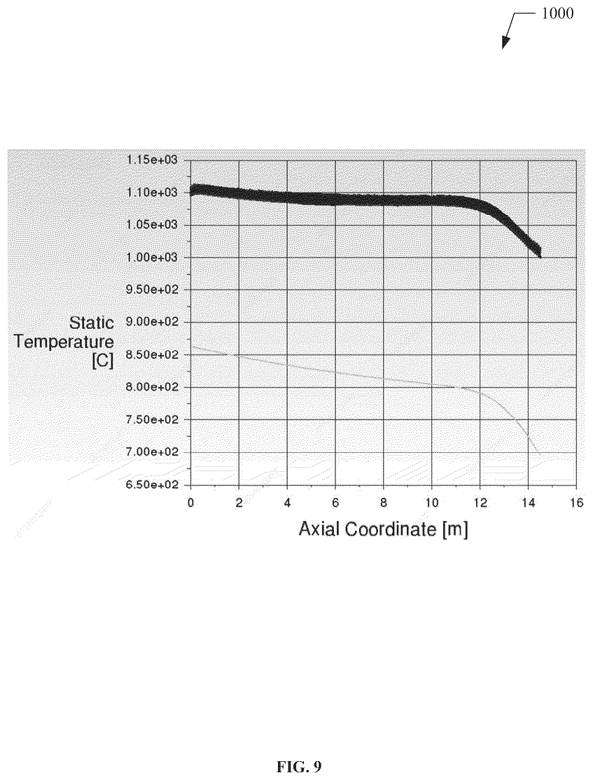

Referring to FIG. 8, a schematic diagram 900 shows a cutaway side view of another example of a furnace having a design similar in some respects to the one described herein with respect to FIGS. 5A and 5B. The example furnace design depicted in FIG. 8 includes multiple process tubes 918 disposed between two walls (only one of which is shown) of a conductive refractory material 902 to prevent process vapor or fluid from directly contacting the conductive refractory material 902.

In the example of FIG. 8, the multiple process tubes 918 correspond to eight different process tubes arranged in a single row, where the single row is situated between the two walls of the conductive refractory material 902. Each of the individual process tubes 918 were modeled with the following properties: a tube inner diameter (ID) of 50.8 mm; a tube length of 14.5 m; and a tube wall thickness of 3.5 mm. The overall surface area of the outer surface of all of the process tubes 918 exposed to radiative heating between the two walls of the conductive refractory material 902 corresponded to 23.6 meters squared.

The example furnace design depicted in FIG. 8 was modeled for an ethane (C₂H₆) steam cracking process. Power 908 provided to the conductive refractory material 902 corresponded to an energy input of 0.098 MW for each of the individual process tubes 918. Tube inlet feed conditions are depicted below in Tables 4 and 5. Tube outlet conditions (corresponding to variables averaged for each of the individual process tubes 918) are depicted below in Table 6.

TABLE 4

| | |
|---|---|
| Temperature (° C.) | 692 |
| Flow rate (kg/s) per tube | 0.033 |
| Pressure (Pascals) | 200000 |

TABLE 5

| Species: | Mass fraction: |
|---|---|
| C2H6 | 0.714 |
| C2H4 | 0 |
| H2 | 0 |
| H2O | 0.286 |

TABLE 6

| | |
|---|---|
| Temperature (K) | 1136.8013 |
| Wall Temperature (K) | 1369.512 |
| Nunnselt Number | 169.84814 |
| Wall Heat Flux (W/m2) | −48948.92 |
| Velocity (m/s) | 57.876856 |
| Density (kg/m3) | 0.37597531 |
| Pressure (Pascals) | 200000 |
| Mass fraction of C2H6 | 0.20950502 |
| Mass fraction of C2H4 | 0.47067298 |
| Mass fraction of H2 | 0.033822 |
| Mass fraction of H2O | 0.286 |

Based on a comparison of the initial mass fraction of ethane (C₂H₆) as shown in Table 5 to the mass fraction of ethane as shown in Table 6, the ethane cracking process modeling with the furnace design depicted in FIG. 8 resulted in an ethane conversion rate of about 71 percent. With respect to thermal data, flux at the heating wall corresponded to 33.44 kW/m2 (averaged based on wall area); and flux at the tube wall corresponded to about 49.2 kW/m2 (averaged based on identifiers associated with each of the individual process tubes 918). With respect to wall temperature data, a maximum wall temperature modeled was about 1210.6° C. and an average wall temperature measurement was about 1189° C. FIG. 9 is a graph 1000 depicting tube temperature profiles, where the top line (“thick”) corresponds to a temperature at the outer diameter (T_OD) of the process tubes 918, a middle line (roughly paralleling the bottom edge of the thick top line but obscured from view in FIG. 9) corresponds to a temperature at the inner diameter (T_ID) of the process tubes 918, and the bottom line corresponds to a temperature of the process gas (T_gas) within the process tubes 918. The “thickness” of the top line in the graph 1000 depicted in FIG. 9 is because there are a range of temperatures around the circumference of each of the individual process tubes 918, with the hottest temperatures facing the heating walls and with the coolest temperatures facing adjacent tube(s).

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present systems are not limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A heating apparatus comprising:
a conductive refractory material without separate heating elements; and
a furnace for heating hydrocarbons, wherein the furnace comprises one or more process tubes extending through an interior of the furnace and configured to receive a process vapor or fluid such that the process vapor or fluid does not contact the conductive refractory material;
wherein the conductive refractory material is at least partially exposed to an interior of the furnace and is configured to receive electrical power from a power source and to generate heat such that the conductive refractory material radiates the heat to the interior of the furnace.

2. The apparatus of claim 1, wherein the conductive refractory material comprises a ceramic material.

3. The apparatus of claim 1, wherein the one or more process tubes are spaced from the conductive refractory material such that the process vapor or fluid does not contact the conductive refractory material.

4. The apparatus of claim 3, wherein the one or more process tubes comprise a plurality of process tubes.

5. The apparatus of claim 4, wherein the plurality of process tubes are arranged in rows of two or more process tubes and said rows alternate with the conductive refractory material.

6. The apparatus of claim 4, wherein the plurality of process tubes are arranged in a single row of multiple process tubes.

7. The apparatus of claim 4, wherein each individual process tube of the plurality of process tubes is separated from the conductive refractory material by at least a process tube wall thickness corresponding to a difference between a process tube outer diameter and a process tube inner diameter.

8. The apparatus of claim 7, wherein the conductive refractory material is shaped as cylinders, plates, or rods.

9. The apparatus of claim 7, wherein the conductive refractory material forms channels, wherein the one or more process tubes include a plurality of process tubes, and wherein each of the plurality of process tubes is disposed within a respective one of the channels.

10. The apparatus of claim 9, wherein the one or more process tubes of the furnace are configured to receive the process vapor or fluid associated with a process selected from the group consisting of: steam cracking; steam methane reforming for syngas production; reforming for ammonia, hydrogen, or methanol; dehydrogenation of propane to propylene; and tar cracking.

11. The apparatus of claim 10, wherein the electrical power from the power source corresponds to a voltage greater than 500 volts.

12. A method of operating a chemical process, the method comprising:
providing a furnace with a conductive refractory material that is at least partially exposed to an interior of the furnace, the conductive refractory material without separate heating elements,
wherein the furnace comprises one or more process tubes extending through the interior of the furnace and configured to receive a process vapor or fluid such that the process vapor or fluid does not contact the conductive refractory material; and
applying electricity directly to the conductive refractory material such that electrical current flowing through the conductive refractory material generates thermal energy to increase the temperature of the conductive refractive material and heat a process vapor or fluid in the process tubes.

13. The method of claim 12, wherein the one or more process tubes are spaced from the conductive refractory material such that the process one or more process tubes do not contact the conductive refractory material, and wherein the chemical process is selected from the group consisting of: a process selected from the group consisting of: steam cracking; steam methane reforming for syngas production; reforming for ammonia, hydrogen, or methanol; dehydrogenation of propane to propylene; and tar cracking.

14. The method of claim 13, wherein the chemical process is a steam methane reforming process, wherein the one or more process tubes comprise a plurality of process tubes, and wherein each individual process tube of the plurality of process tubes is separated from the conductive refractory material by at least a process tube wall thickness corresponding to a difference between a process tube outer diameter and a process tube inner diameter.

15. The method of claim 13, wherein the chemical process is a steam cracking process corresponding to an ethane cracking process or a steam cracking for olefins process, wherein the one or more process tubes comprise a plurality of process tubes, and wherein each individual process tube of the plurality of process tubes is separated from the conductive refractory material by at least a process tube wall thickness corresponding to a difference between a process tube outer diameter and a process tube inner diameter.

16. The apparatus of claim 10, wherein the electrical power from the power source corresponds to a voltage greater than 1000 volts.

17. The apparatus of claim 10, wherein the electrical power from the power source corresponds to a voltage greater than 2000 volts.

18. The apparatus of claim 1, wherein the conductive refractory material is configured to operate at temperatures up to 2000° C. or greater.

19. The method of claim 12, wherein the conductive refractory material is shaped as cylinders, plates, or rods.

20. The method of claim 15, wherein the conductive refractory material forms channels, and wherein each of the plurality of process tubes is disposed within a respective one of the channels.
